(19) United States
(12) Patent Application Publication
(10) Pub. No.: US 2024/0374499 A1
(43) Pub. Date: Nov. 14, 2024

(54) COSMETIC FRAGRANCE COMPOSITION IN THE FORM OF AN ALCOHOL-FREE EMULSION

(71) Applicant: CAPSUM, Marseille (FR)

(72) Inventors: Elodie GALANDRIN, Plan De Cuques (FR); Mathieu GOUTAYER, Saint Malo (FR)

(21) Appl. No.: 18/658,601

(22) Filed: May 8, 2024

(30) Foreign Application Priority Data
May 10, 2023 (FR) 23 04643

(51) Int. Cl. A61K 8/73; A61Q 13/00

(52) U.S. Cl. CPC A61K 8/731; A61Q 13/00; A61K 2800/30; A61K 2800/412

(57) ABSTRACT

A composition, in particular cosmetic composition, in the form of an oil-in-water emulsion comprising at least one fatty phase in the form of drops dispersed in a continuous aqueous phase, the fatty phase and the aqueous phase being immiscible with one another at room temperature and atmospheric pressure, characterised in that: a) the continuous aqueous phase comprises at least microcrystalline cellulose, and b) the dispersed fatty phase comprises at least 5%, preferably at least 10%, in particular at least 20% or even at least 30%, by weight of perfume(s) relative to the total weight of the composition.

This application claims priority to French Patent Application 23 04643 filed May 10, 2023, the entire disclosure of which is incorporated by reference herein.

The present invention concerns a new alcohol-free cosmetic fragrance composition which may, for example, be an eau de toilette, an eau de Cologne, a perfume extract, a perfume spirit or a perfume water, depending on the perfume content it contains and the notes it expresses.

This fragrance composition has excellent properties, such as kinetic stability and the ability to be sprayed in the form of fine particles, as well as the absence of a sticky sensation to the touch, and even the olfactory fidelity of the fragrance and the persistence of the fragrant note over time after application.

There is growing consumer demand for alcohol-free fragrances, mainly because alcohol is not good for the skin and skin irritations can occur with other cosmetic products containing alcohol.

So there is a consumer need for alcohol-free fragrance products.

However, alcohol, such as ethanol, plays an important role in the formulation of perfumes because of its great capacity to dissolve them.

Many approaches have been explored in order to prepare suitable alcohol-free cosmetic fragrances. For example, one approach is based on oil-in-water emulsified compositions comprising surfactants, in particular non-ionic surfactants, in high concentrations (generally more than 10% by mass), to dissolve fragrances (WO 2017/059513 and CN 103637942). However, this approach is not satisfactory insofar as the high quantities of surfactants required to dissolve the perfumes (i) induce a sticky character which alters the sprayability, (ii) affect the olfactory quality of the perfumes and (iii) may lead to stability problems.

As a result, there is a need for an alcohol-free fragrance formula that offers high sprayability while maintaining fragrance stability and olfactive properties.

The aim of the present invention is therefore to provide a cosmetic fragrance composition in the form of an oil-in-water emulsion (or “direct emulsion”) which contains very low amounts of alcohol, or no alcohol at all, while guaranteeing the stability of the emulsion over time, satisfactory or even excellent sprayability, and olfactory stability over time.

In particular, one aim of the present invention is to provide a cosmetic fragrance composition which allows high sprayability, while retaining the same quality and properties as conventional alcohol-based perfuming compositions, such as olfactory properties and long-term stability.

This invention relates to a composition, in particular a cosmetic composition, in the form of an oil-in-water emulsion comprising at least one fatty phase in the form of drops dispersed in a continuous aqueous phase, the fatty phase and the aqueous phase being immiscible with one another at room temperature and atmospheric pressure, characterized in that:
a) the continuous aqueous phase comprises at least microcrystalline cellulose, and
b) the dispersed fatty phase comprises at least 5%, preferably at least 10%, in particular at least 20% or even at least 30%, by weight of perfume(s) relative to the total weight of the composition.

Preferably, in a composition according to the invention, the dispersed fatty phase also comprises at least one lipophilic cationic compound comprising at least one primary, secondary, tertiary or quaternary amine function and/or the continuous aqueous phase also comprises at least one non-ionic surfactant.

Against all expectations, the inventors have observed that the presence of microcrystalline cellulose makes it possible to obtain a cosmetic fragrance composition in the form of a stable oil-in-water emulsion (or “direct emulsion”) in the presence of very small quantities of alcohol, or even no alcohol at all, while guaranteeing the stability of the emulsion over time, satisfactory or even excellent sprayability, and olfactory stability over time. The invention also makes it possible to obtain fragrance compositions that do not feel sticky to the touch.

Stability can be assessed with the naked eye, and can be considered as the absence of creaming and/or the absence of phase shift between the dispersed fatty phase and the continuous aqueous phase.

For the purposes of this invention, the term “emulsion” may be used interchangeably with the term “dispersion”.

According to the invention, the pH of a composition is typically between 4.0 and 8.0, in particular between 5.0 and 7.0.

Unless otherwise stated, all the following is considered to be at room temperature (e.g. T=25° C.±2° C.) and atmospheric pressure (760 mm Hg, i.e. 1.013*10⁵ Pa or 10¹³ mbar).

Preferably, a composition according to the invention is not in the form of a nanoemulsion and/or does not comprise a high amount of surfactant(s), in particular non-ionic surfactants.

In particular, a composition according to the invention comprises less than 10%, preferably less than 5%, by weight of surfactant(s) relative to the total weight of the composition.

Preferably, a composition according to the invention is in the form of a microemulsion.

A composition according to the invention does not comprise (or is not based on) granules obtained by spray-drying. A composition according to the invention is not in the form of and/or does not comprise spray-dried solid granules.

The present invention is not based on spraying an emulsion onto microcrystalline cellulose particles.

A composition according to the invention is not in the form of a cross-linked hydrophilic matrix or does not comprise a cross-linked hydrophilic matrix.

A composition according to the invention is not in the form of matrix particles comprising a cross-linked polymer or does not comprise matrix particles comprising a cross-linked polymer, in particular derived from the group consisting of alginates and pectins.

### Continuous Aqueous Phase

Advantageously, a composition according to the invention, in particular the continuous aqueous phase, is not solid at ambient temperature and ambient pressure, i.e. it is capable of flowing under its own weight.

According to the invention, the continuous aqueous phase comprises at least water. In addition to distilled or deionised water, water suitable for the invention may also be natural spring water or floral water.

According to one embodiment, the percentage by weight of water in the continuous aqueous phase is at least 30%, preferably at least 40%, in particular at least 50%, and better still at least 60%, in particular between 70% and 98%, and preferably between 75% and 95%, relative to the total mass of the continuous aqueous phase.

According to the invention, the continuous aqueous phase comprises at least microcrystalline cellulose, in particular as defined below.

According to the invention, the continuous aqueous phase may also comprise at least one non-ionic surfactant, in particular as defined below.

Preferably, a composition, in particular the continuous aqueous phase, does not comprise carbomer (or acrylic polymer).

Preferably, a composition, in particular the continuous aqueous phase, does not comprise any base, in particular alkali metal hydroxide, and in particular NaOH (or sodium hydroxide).

Preferably, the composition according to the invention, and in particular the continuous aqueous phase, has a high-shear viscosity, as measured at 25° C. and under a shear stress of 100 s⁻¹, of less than or equal to 150 mPa·s, preferably less than or equal to 100 mPa·s, in particular less than or equal to 90 mPa·s, better less than or equal to 80 mPa·s, and preferably between 50 mPa·s and 150 mPa·s, particularly between 60 mPa·s and 135 mPa·s, and especially between 70 mPa·s and 90 mPa·s.

Preferably, a composition according to the invention is sprayable.

By “sprayable”, (“pulvérisation” or “pulvérisable” in French), within the meaning of the invention, we mean the ability of a composition to be sprayed in the form of fine droplets using spray-type delivery devices (pumps) or even pressurisation devices (aerosols). In other words, a composition according to the invention is advantageously a sprayable composition.

Advantageously, the composition according to the invention, in particular the continuous aqueous phase, has a flow threshold value of less than or equal to 10 Pa, in particular less than or equal to 8 Pa, very particularly less than or equal to 5 Pa, and in particular less than or equal to 1 Pa.

Advantageously, the composition according to the invention, in particular the continuous aqueous phase, has a flow threshold value of between 0.05 Pa and 10 Pa, preferably between 0.5 Pa and 8 Pa, and more preferably between 1 Pa and 5 Pa.

The viscosity and pour point are measured using the following method:

All the measurements were carried out using a Ta instrument DHR10 rheometer fitted with a 40 mm diameter mobile forming a 1° cone—the measurement air gap being 29 μm. The rheometer is controlled by Trios software. Measurements were taken at 18° C., the temperature being controlled by a Peltier device. Rheological behaviour is measured here using a flow sweep protocol. Once the sample is in place and the temperature of 18° C. has been reached, the sample is left to rest for 60 seconds so that it can relax its stresses. It is then subjected to a logarithmic shear scan between 0.01 and 500 s⁻¹ with 5 points per decade. The viscosity (mPa·s) and stress (Pa) curves are plotted using Trios software. Analysis of the stress curve by the software, applying the Herschel-Bulkley model, enables the rheological behaviour to be expressed according to the following equation.

τ=τ₀+k\dot{γ}ⁿ,siτ≥τ₀
where τ is the shear stress; τ₀ is the flow threshold value; k is the consistency coefficient; \dot{γ} is the shear rate and n is the flow index.

A composition according to the invention satisfying the viscosity and pour point values described above presents a non-obvious compromise between kinetic stability of the emulsion, liquid character and sprayability. This compromise is made possible by the presence of microcrystalline cellulose in the continuous aqueous phase.

### Microcrystalline Cellulose

Microcrystallin cellulose is generally prepared from wood pulp: the said pulp is cut into small particles which are chemically hydrolysed, in particular with a mineral acid, followed by a filtration and drying stage.

According to a first variant, the microcrystalline cellulose may be in particle form. Preferably, the microcrystalline cellulose has a particle size defined by a median diameter D50 ranging from 5 to 140 μm.

Preferably, the microcrystalline cellulose is chosen from microcrystalline cellulose with a median diameter of less than 30 μm and preferably 10 μm. Alternatively, the microcrystalline cellulose preferably has a median diameter of 130 μm.

The median diameter is a parameter that is well known to those skilled in the art and can be measured using techniques that are familiar to them, such as laser granulometry or selective sieving.

Such microcrystalline cellulose is marketed in particular by FMC Biopolymers under the name Avicel or by the company JRS.

According to a second variant, the microcrystalline cellulose can be in the form of a fibre, preferably with an average diameter of 2 to 5 nm. Average fibre diameter means the average diameter of the fibres by volume.

Such microcrystalline cellulose is marketed by IWASE COSFA under the name Rheocrysta C-2SN.

According to a third variant, the microcrystalline cellulose may be in the form of a mixture (or association or coupling) with a natural gum, in particular a cellulose or xanthan gum. This can be referred to as co-processed (or functionalised) microcrystalline cellulose; in particular, microcrystalline cellulose to which a natural gum has been bonded (or grafted).

This type of co-processed microcrystalline cellulose is marketed by CFF GmbH & Co KG under the name Sensocel stab 026 or Sensocel stab 026N.

Preferably, the microcrystalline cellulose is a co-processed microcrystalline cellulose, preferably with a natural gum, in particular a cellulose or xanthan gum, and especially a cellulose gum.

A dispersion according to the invention may comprise from 0.1% to 10%, preferably from 0.5% to 5%, advantageously from 1% to 2.5% by weight of oil(s) relative to the total weight of said dispersion.

### Natural Gum

According to the invention, the continuous aqueous phase may also comprise at least one natural gum.

As can be seen from Example 1, such a compound is advantageous in that it provides an alcohol-free perfume formula with further improved properties in terms of sprayability, stability and olfactory properties of the perfumes.

Preferably, a natural gum is chosen from cellulose, starch, xanthan, inulin, gellan, pullulan, curdlan, gum arabic, amylopectin, dextran, hemicellulose and mixtures thereof, in particular cellulose and/or xanthan, and especially cellulose.

According to a particular embodiment, a composition according to the invention does not comprise xanthan. Preferably, the natural gum likely to be present in the continuous aqueous phase is not a xanthan.

According to a first variant, the natural gum is a free raw material, i.e. used as such. In other words, the natural gum is not in a form associated (or coupled) with another raw material, in particular microcrystalline cellulose.

According to another variant, the natural gum is used in a form associated (or coupled) with another raw material, and in particular with microcrystalline cellulose. In other words, in this variant, such a natural gum is not a free raw material. Such a coupled form is advantageous in that it improves the incorporation of microcrystalline cellulose into the continuous aqueous phase.

Preferably, a composition according to the invention may comprise from 0.1% to 10%, preferably from 0.2% to 5%, and more preferably from 0.3% to 2.5%, by weight of natural gum relative to the total weight of the continuous aqueous phase.

### Non-Ionic Surfactants

According to the invention, the continuous aqueous phase may also comprise at least one non-ionic surfactant.

As can be seen from Example 2, such a compound is advantageous in that it provides an alcohol-free perfume formula with high sprayability while maintaining the stability and olfactory properties of the perfumes.

Non-ionic surfactants are used in the continuous aqueous phase and are therefore hydrophilic. All or some of the non-ionic surfactants can naturally be placed at the water/oil interface due to their intrinsic amphiphilic properties.

Non-ionic surfactants are known compounds in their own right (for example, on this subject, “Handbook of Surfactants”, by M. R. Porter, Blackie & Son Publishers (Glasgow and London), 1991, pages 116-178.

Preferably, the non-ionic surfactants are chosen from those with an HLB value of between 5 and 15, in particular between 7 and 13, and better still between 8 and 10.

The non-ionic surfactant is preferably chosen from the group consisting of monooxyalkylenation or polyglycerol non-ionic surfactants. The oxyalkylene unit is, more specifically, an oxyethylene or oxypropylene unit, or a combination thereof, preferably an oxyethylene unit

Preferably, the non-ionic surfactant is a polyglycerol surfactant, in particular Polyglyceryl-3 Distearate marketed by BASF Care Creations under the name CREMOPHOR GS32 or Polyglyceryl-10 Stearate marketed by BASF Care Creations under the name Emulgade Verde 10 MS.

Preferably, a composition according to the invention can comprise 0.1% to 10%, preferably 0.5% to 5%, and in particular 1% to 5%, by weight of non-ionic surfactant(s), relative to the total weight of the continuous aqueous phase.

### Alcohols

The continuous aqueous phase of a composition according to the invention advantageously comprises less than 10%, preferably less than 5%, in particular less than 2%, better still less than 1%, and very particularly less than 0.5%, or is even free, of alcohol(s), in particular ethanol.

By “alcohol” we mean in particular a lower alkyl alcohol. A lower alkyl alcohol can be an aliphatic monoalcohol comprising from 2 to 6 carbon atoms. Such a lower alkyl alcohol can be ethanol.

The composition is preferably free of any lower alkyl alcohol.

Within the meaning of the present invention, “ethanol-free” or “alcohol-free” may also refer to a cosmetic which is prepared by a preparation process preferably comprising no step of adding a lower alkyl alcohol and no step of mixing an alkyl alcohol with other ingredients.

However, it cannot be ruled out that traces of lower alkyl alcohol, in particular traces of ethanol, may be present in some of the ingredients used to prepare the composition according to the invention, in particular in certain perfumes.

### Acids

According to a particular embodiment, the continuous aqueous phase may also comprise at least one acid, preferably chosen from citric acid, polyacrylic acid, phytic acid, phosphoric acid, polyaspartic acid, tartaric acid, malic acid, gluconic acid, or more generally a polyacid, and mixtures thereof, and particularly citric acid.

Such a method is advantageous in that it makes it possible to improve the kinetic stability of a composition further according to the invention.

### Dispersed Fatty Phase

A composition according to the invention comprises a dispersed fatty phase (or oily phase).

For obvious reasons, the continuous aqueous phase and the dispersed fatty phase are immiscible. “Immiscible” or “not immiscible” within the meaning of the present invention means that the solubility of a first phase in a second phase is advantageously less than 5% by mass, and vice versa.

A composition according to the invention may comprise from 5% to 50%, preferably from 10% to 40%, and in particular from 20% to 30%, by weight of fatty phase relative to the total weight of the composition.

For the purposes of this invention, the fatty phase is in the form of drops, which may be referred to interchangeably as “drops (G1)”.

In a particular embodiment, some or all of the drops G1 may be microscopic. For the purposes of this invention, the term “microscopic” or “microscopic drop” refers to drops of dispersed phase that are not visible to the naked eye. According to this particular method, the dispersed fatty phase droplets advantageously have a diameter of less than 250 μm, preferably a diameter of less than 125 microns, in particular a diameter of less than 75 μm.

In another particular embodiment, some or all of the drops G1 may be macroscopic. For the purposes of this invention, “macroscopic” or “macroscopic droplet” refers to droplets of dispersed phase visible to the naked eye. Thus, according to this particular embodiment, the drops have a diameter greater than or equal to 250 μm, in particular greater than or equal to 500 μm, preferably greater than or equal to preferably greater than or equal to 750 μm, or even greater than or equal to 1,000 μm, or even between 250 μm and 3,000 μm, better still between 500 μm and 2,000 μm, and in particular between 750 μm and 1,000 μm

Determining the size (or diameter) of the drops is a matter for the general knowledge of the person skilled in the art.

### Fragrances

The dispersed fatty phase comprises at least one fragrance.

For obvious reasons, a perfume according to the invention is a lipophilic agent, i.e. soluble or dispersible in an organic solvent, in particular an oil.

A “perfume”, also indifferently designated by “perfuming agent”, “perfume juice” or “perfume concentrate” within the meaning of the present invention, may be chosen from compounds having the INCI name “Parfum” or “Fragrance”, and mixtures thereof. The term “perfume” as used in this invention also includes essential oils.

For the purposes of this invention, the term “perfume” does not mean a mixture comprising a perfume concentrate and alcohol.

The perfumes that can be used according to the invention are ingredients commonly used in perfumery. Their nature does not call for a more detailed description here, which would not be exhaustive, as the craftsman is able to choose them on the basis of his general knowledge and according to the desired olfactory effect. These fragrances belong to chemical classes as varied as alcohols, aldehydes, ketones, esters, ethers, acetates, nitriles, terpene hydrocarbons, nitrogenous or sulphurous heterocyclic compounds, as well as essential oils of natural or synthetic origin. Many of these ingredients are also listed in reference texts such as the book by S. Arctander, Perfume and Flavor Chemicals, 1969, Montclair, New Jersey, USA, or more recent versions, or in other works of a similar nature, as well as in more recent scientific and patent literature relating to the art of perfumery.

For example, a perfume is a compound or mixture of compounds that is at least partially volatile at room temperature and whose odour can be detected. A fragrance made up of essential oils is generally diluted in order to express its full olfactory potential, i.e. a perception that evolves over the course of the day after application to the surface to be treated, thanks to the presence of several fragrant organic compounds with different volatilities. The creation of a perfume involves combining several fragrance raw materials to give the perfume composition a top note, a middle note and a base note.

A perfume can be prepared from natural or synthetic organic fragrance materials.

Examples of natural fragrance materials are extracts of flowers, leaf stems, fruit, bark, roots, wood, herbs, grasses, resins, balms and mixtures thereof.

These vegetable fragrances may be essential oils such as bergamot, rose, lavender, sandalwood, cardamom, sage, chamomile, clove, meadowfoam, mint, cinnamon leaf, juniper, vetiver, olibanum, galbanum, labdanum, and mixtures thereof.

Examples of perfuming materials of synthetic origin are hedione, ethylene brassilate, habanolide, benzyl acetate, benzyl benzoate, phenoxyethyl isobutyrate, p-tert-butylcyclohexyl acetate, citronellyl acetate, citronellyl formate, geranyl acetate, acetatetate, citronellyl acetate, citronellyl formate, geranyl acetate, linalyl acetate, dimethylbenzylcarbinyl acetate, phenylethyl acetate, linalyl benzoate. benzyl formate, alkylcyclohexyl ethyl methyl phenyl glycinate propionate, styralyl propionate and benzyl salicylate, benzylethyl ether, linear alkanals with 8 to 18 carbon atoms, citral, citronellal, citronellyloxyacetaldehydehyde, cyclamenaldehyde, hydroxycitronellal, ionones such as alpha-isomethylionone, methylcedrylketone, anethol, citronellol, eugenol, isoeugenol, geraniol, linalool, phenylethyl alcohol, terpineol, terpenes, and mixtures thereof.

These compounds often take the form of a mixture of two or more of these odorous substances.

The characteristics of a composition according to the invention are such that they allow high contents of perfume(s), without prejudice to the kinetic stability and sprayability of the composition.

The dispersed fatty phase thus comprises at least 5%, preferably at least 10%, in particular at least 20% or even at least 30%, by weight of perfume(s) relative to the total weight of the composition, in particular as defined below.

In particular, the dispersed fatty phase of a composition according to the invention can advantageously comprise from 5% to 30%, and preferably from 10% to 20%, by weight of perfume(s) relative to the total weight of the composition.

Also, the composition can advantageously comprise from 10% to 100%, preferably from 20% to 99.95%, in particular from 30% to 99%, or even from 40% to 90%, and very particularly from 50% to 80%, by weight of fragrance(s) relative to the total weight of the dispersed fatty phase.

### Lipophilic Cationic Compounds

According to the invention, the dispersed fatty phase can also comprise at least one lipophilic (or fat-soluble) cationic compound comprising at least one primary, secondary, tertiary or quaternary amine function.

As can be seen from Example 2, such a compound is advantageous in that it provides an alcohol-free perfume formula with high sprayability while maintaining the stability and olfactory properties of the perfumes.

Unless otherwise stated, “lipophilic cationic compound”, “cationic-type compound” or “cationic compound” means a compound containing chemical functions of the cationic type.

In the context of this application, and unless otherwise stated, “cationic-type chemical function” means a chemical B function capable of taking up a proton to give a BH⁺ function. Depending on the conditions of the environment in which it is found, the cationic compound therefore contains chemical functions in the B form, or in the BH⁺ form, its conjugate acid.

Examples of cationic chemical functions are ammonium, sulphonium or phophonium, preferably ammonium.

A lipophilic cationic compound comprises at least one primary, secondary, tertiary or quaternary amine function, preferably present in the form of ammonium cations.

The lipophilic cationic compound comprising at least one primary, secondary, tertiary or quaternary amine function may be chosen from at least one polymer modified with a primary, secondary, tertiary or quaternary amine function, a cationic surfactant, and mixtures thereof.

Preferably, a composition according to the invention can comprise from 0.05% to 10%, preferably from 0.1% to 5%, and better still from 0.2% to 1%, by weight of lipophilic cationic compound(s) comprising at least one primary, secondary, tertiary or quaternary amine function relative to the total weight of the dispersed fatty phase.

Polymer Modified with a Primary, Secondary, Tertiary or Quaternary Amine Function

In the context of the present application, a polymer modified by a primary, secondary, tertiary or quaternary amine function may be indifferently defined as “lipophilic cationic polymer”, “cationic-type polymer” or “cationic polymer”, “lipophilic cationic polymer”, “cationic-type polymer” or “cationic polymer”.

An example of a cationic polymer is any polymer formed by the polymerisation of monomers at least some of which carry cationic chemical functions, such as primary, secondary, tertiary or quaternary amine functions.

Examples of such monomers are aziridine or any ethylenically unsaturated monomer containing at least one primary, secondary, tertiary or quaternary amine function.

Examples of cationic polymers suitable for implementing the invention include amodimethicone, derived from a silicone polymer (polydimethylsiloxane, also known as dimethicone), modified with primary and secondary amine functions.

Mention may also be made of amodimethicone derivatives, such as amodimethicone copolymers, aminopropyl dimethicone and, more generally, linear or branched silicone polymers containing amine functions.

Examples include bis-isobutyl PEG-14/amodimethicone copolymer, Bis(C13-15 Alkoxy) PG-Amodimethicone, Bis-Cetearyl Amodimethicone and bis-hydroxy/methoxy amodimethicone.

Preferably, a cationic polymer is a silicone polymer modified with a primary, secondary, tertiary or quaternary amine function, such as amodimethicone.

According to a preferred embodiment, the cationic-type polymer is amodimethicone, in particular as described in WO2019002308, or a compound of formula (I) or of formula (IV) described in the patent application filed under No. FR2205166.

### Cationic Surfactants

A cationic surfactant according to the invention is lipophilic and may be chosen from any lipophilic cationic surfactant known to the person skilled in the art.

Preferably, the cationic surfactant is chosen from those marketed by SurfactGreen under the names CosmeGreen ES1822+ (INCI: Arachidyl/Behenyl Betainate Esylate (and) Arachidyl/Behenyl Alcohol) or CosmeGreen MB1618 (Cetearyl Alcohol (and) Cetearyl Betainate Mesylate), or from those marketed by Inolex under the name KerazyneMB (INCI: Polyester-11), and mixtures thereof.

### Oils

According to a particular embodiment, the dispersed fatty phase of a composition according to the invention may also comprise at least one oil.

By “oil” we mean a fatty substance that is liquid at room temperature.

Examples of oils that can be used in a dispersion of the invention include:
hydrocarbon oils of vegetable origin, such as hydrogenated jojoba oil, hydrogenated sunflower oil, hydrogenated castor oil and hydrogenated coconut oil;
hydrocarbon oils of animal origin, such as perhydrosqualene and squalane;
synthetic esters and ethers, in particular of fatty acids, such as oils of formulae R1COOR2 and R1OR2 in which R1 represents the residue of a C8 to C29 fatty acid and R2 represents a branched or unbranched C3 to C30 hydrocarbon chain, such as Purcellin oil, isononyl isononanoate, isodecyl neopentanoate, isopropyl myristate, ethyl-2-hexyl palmitate, octyl-2-dodecyl stearate, octyl-2-dodecyl erucate and isostearyl isostearate; hydroxy esters such as isostearyl lactate, octylhydroxystearate, octyldodecyl hydroxystearate, diisostearyl malate, triisocetyl citrate, fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetrabenate (DUB PTB) or pentaerythrityl tetraisostearate (Prisorine 3631);
linear or branched hydrocarbons of mineral or synthetic origin, such as volatile or non-volatile paraffin oils and their derivatives, petroleum jelly, polydecenes, hydrogenated polyisobutene such as Parléam oil;
silicone oils, such as, for example, volatile or non-volatile polymethylsiloxanes (PDMS) with a linear or cyclic silicone chain, which are liquid or pasty at room temperature, in particular cyclopolydimethylsiloxanes (cyclomethicones) such as cyclohexasiloxane and cyclopentasiloxane polydimethylsiloxanes (or dimethicones) containing alkyl, alkoxy or phenyl groups, during or at the end of the silicone chain, groups having from 2 to 24 carbon atoms; phenylated silicones such as phenyltrimethicones, phenyldimethicones, phenyltrimethylsiloxydiphenylsiloxanes, diphenyl-dimethicones, diphenylmethyldiphenyl trisiloxanes, 2-phenylethyltrimethyl-siloxysilicates, and polymethylphenylsiloxanes;
fatty alcohols with 8 to 26 carbon atoms, such as cetyl alcohol, stearyl alcohol and their mixture (cetyl stearyl alcohol), or octyldodecanol;
Partially hydrocarbon and/or silicone fluorinated oils as described in JP-A-2-295912;
and mixtures thereof.

According to a particular embodiment, the dispersed fatty phase may comprise at least one oil having a density at 20° C. of at least 0.90 g/cm3, preferably a density between 0.95 g/cm3 and 1.10 g/cm3. Such a method is advantageous in that it makes it possible to improve the kinetic stability of a composition further according to the invention.

Preferably, the fatty phase does not comprise silicone oil, and preferably does not comprise polydimethylsiloxane (PDMS).

Of course, the person skilled in the art will take care to choose any oils and/or their quantity in such a way that the advantageous properties of a composition according to the invention are not or not substantially altered by the envisaged addition. These adjustments are a matter for the general knowledge of the person skilled in the art.

According to one embodiment, a composition according to the invention comprises from 1% to 30%, preferably from 2% to 20%, and better still from 5% to 10%, by weight of oil(s) relative to the total weight of the dispersed fatty phase.

### Additional Compound(s)

According to the invention, a composition according to the invention, in particular the continuous aqueous phase and/or the dispersed fatty phase, may also comprise at least one additional compound other than the perfumes, microcrystalline cellulose, hydrophilic nonionic surfactants, lipophilic cationic compounds, natural gums and oils mentioned above.

A composition according to the invention may also comprise powders; reflective particles, in particular flakes, in particular as defined in FR3082731; colouring agents, in particular chosen from water-soluble or insoluble, fat-soluble or insoluble, organic or inorganic colouring agents, optical effect materials, liquid crystals and mixtures thereof; fillers, in particular as described in FR1755907; pigments; nacres; emulsifying and/or non-emulsifying silicone elastomer, in particular as described in EP2353577; “soft focus” fillers; texturing agents, in particular polyethylene glycols (e.g. marketed under the name Carbowax) or glycerine; preservatives; humectants; stabilisers; chelating agents; emollients; hydrophilic and/or lipophilic gelling agents, in particular those described below; pH modifiers, osmotic strength modifiers and/or refractive index modifiers etc., or any customary cosmetic additive; and mixtures thereof.

As an additional compound, a composition according to the invention, in particular the continuous aqueous phase and/or the dispersed fatty phase, may also comprise at least one biological/cosmetic active ingredient, chosen in particular from moisturising agents, healing agents, depigmenting agents, UV filters, desquamating agents, antioxidant agents, active ingredients stimulating the synthesis of dermal and/or epidermal macromolecules, dermodecontracting agents, antiperspirant agents, soothing agents and/or anti-ageing agents, and mixtures thereof. Such assets are described in particular in FR1558849.

As an additional compound, a composition according to the invention, in particular the continuous aqueous phase and/or the dispersed fatty phase, may also comprise at least one hair active ingredient, in particular as described in the patent application filed under No. FR2206462.

Of course, the person skilled in the art will take care to choose any additional compounds and/or their quantity in such a way that the advantageous properties of a composition according to the invention are not or not substantially altered by the envisaged addition. These adjustments are a matter for the general knowledge of the person skilled in the art.

According to a particular embodiment, a composition according to the invention does not comprise a repellent, in particular chosen from ethyl 3-(N-n-butyl-N-acetylamino) propionate. According to a particular embodiment, a composition according to the invention does not comprise a repellent function.

According to a particular embodiment, a composition according to the invention does not comprise alginate or a salt thereof or a derivative thereof.

According to a particular embodiment, a composition according to the invention does not comprise clay, in particular does not comprise smectite, bentonite, kaolinite, illite, chlorite, vermiculite, sepiolite and/or attapulgite.

### Lipophilic Gelling Agent

A lipophilic gelling agent, i.e. one that is soluble or dispersible in the fatty phase, may be chosen from organic or inorganic, polymeric or molecular gelling agents; fats that are solid at ambient temperature and pressure, in particular chosen from waxes, pasty fats, butters, and mixtures thereof, and preferably from polymeric gelling agents. Such lipophilic gelling agents are described in particular in WO2019002308.

Lipophilic gelling agents that can be used in the present invention include esters of dextrin and a fatty acid, such as dextrin palmitates. Examples of dextrin fatty acid esters are dextrin palmitates, dextrin myristates, dextrin palmitates/ethylhexanoates and mixtures thereof. Examples include esters of dextrin and fatty acid(s) marketed under the names Rheopearl® KL2 (INCI name: dextrin palmitate), Rheopearl® TT2 (INCI name: dextrin palmitate ethylhexanoate), and Rheopearl® MKL2 (INCI name: dextrin myristate) by Miyoshi Europe, as well as dextrin palmitate marketed by The Innovation Company.

THIXCIN® R from Elementis Specialties (INCI: Trihydroxystearin), OILKEMIA™ 5S polymer from Lubrizol (INCI: Caprylic/Capric Triglyceride (and) Polyurethane-79), the Oilkemia™ 5S CC polymer, (INCI: INCI: Caprylic/Capric Triglyceride (and) Hydrogenated Poly(C6-20 Olefin) (and) HDI/Trimethylol Hexyllactone Crosspolymer), Estogel M from PolymerExpert (INCI: CASTOR OIL/IPDI COPOLYMER & CAPRYLIC/CAPRIC TRIGLYCERIDE), EMC30 (INCI: Caprylic/Capric Triglyceride (and) Castor Oil/IPDI Copolymer), Hydrogenated Castor Oil/Sebacic Acid Copolymer and derivatives thereof, marketed respectively under the names Estogel Green (or Estogel G) and Estogel Green 40 by PolymerExpert, and mixtures thereof.

Advantageously, a lipophilic gelling agent is a heat-sensitive gelling agent.

### Hydrophilic Gelling Agents

A hydrophilic gelling agent, i.e. soluble or dispersible in water, may be chosen from:
natural gelling agents, in particular chosen from among algae extracts, plant exudates, seed extracts and microorganism exudates, such as alkasealan marketed by the company Hakuto (INCI: Alcaligenes Polysaccharides), and other natural agents, in particular hyaluronic acid,
semi-synthetic gelling agents, in particular chosen from cellulose derivatives and modified starches,
synthetic gelling agents, in particular chosen from homopolymers of (meth)acrylic acid or one of their esters, copolymers of (meth)acrylic acid or one of their esters, copolymers of AMPS (2-acrylamido-2-methylpropanesulphonic acid), associative polymers, of those described in FR2999921,
other gelling agents, in particular selected from clays and glycerine, and
mixtures thereof.

These hydrophilic gelling agents are described in more detail in FR3041251.

Advantageously, a hydrophilic gelling agent is a heat-sensitive gelling agent.

Preferably, a composition according to the invention does not comprise:
lecithin or one of its derivatives, in particular does not comprise hydrogenated lecithin;
polyvinylpyrrolidone;
hydrogenated polyisobutene;
polyglyceryl-6 caprylate/caprate and/or sodium caproyl/lauroyl lactylate; and/or
Caprylic/Capric Triglyceride.

### ISO 16128

Advantageously, a composition according to the invention comprises a percentage of ingredients of natural origin greater than or equal to 95%, preferably greater than or equal to equal to 96%, in particular greater than or equal to 97%, better than or equal to 98%, and very particularly greater than or equal to 99%, according to standard ISO 16128.

In other words, a composition according to the invention is devoid of, or comprises less than 5%, preferably less than 4%, in particular less than 3%, better still less than 1%, and very particularly less than 0.5% by weight, of ingredients of non-natural origin according to standard ISO 16128.

A method for calculating the percentage of ingredients of natural origin according to the ISO 16128 standard is described in WO2022167567.

In a particular embodiment, the continuous aqueous phase may itself be in the form of a direct emulsion.

In a particular embodiment, the dispersed fatty phase may itself be in the form of an inverse emulsion.

### Preparation Method

The preparation of the continuous aqueous phase and the dispersed fatty phase of a composition according to the invention is a matter for the general knowledge of the person skilled in the art.

The preparation of a composition according to the invention is also a matter for the general knowledge of the person skilled in the art. By way of illustration, a composition according to the invention may be obtained by means of non-micro/milli-fluidic manufacturing processes, such as for example by agitation with Rayneri, or by means of micro/milli-fluidic manufacturing processes, for example those described in WO2010063937, FR2964017, WO2012089820, WO2021037999, WO2022106361, EP2292752, EP0525731, EP1020177, EP1025842.

### Uses

Preferably, a dispersion according to the invention can be used directly, after the above-mentioned preparation methods, as a composition, in particular a cosmetic composition.

The compositions according to the invention can therefore be used in particular in the cosmetic field.

They may include, in addition to the above-mentioned ingredients or compounds, at least one physiologically acceptable medium.

The physiologically acceptable medium is generally adapted to the nature of the substrate to which the composition is to be applied, as well as to the appearance in which the composition is to be packaged.

In one embodiment, the physiologically acceptable medium is represented directly by the continuous aqueous phase as described above.

In the context of the invention, and unless otherwise stated, “physiologically acceptable medium” means a medium suitable for cosmetic applications, and suitable in particular for the application of a composition of the invention to keratinous material, in particular the skin and/or hair, and more particularly the skin.

A cosmetic composition according to the invention is advantageously a perfume composition. A cosmetic composition according to the invention may be an eau de toilette, an eau de Cologne, a perfume extract, a perfume spirit, a perfume veil, a perfume water or a perfume water.

Thus, a cosmetic composition according to the invention is advantageously a perfuming composition, in particular an alcohol-free perfuming composition, in liquid form intended to perfume an individual after spraying or application to the skin, hair and/or clothing. Such a product is not rinsed off after application.

A fragrance composition will therefore be distinguished from a perfumed composition. A cosmetic composition can be fragranced without being perfuming.

Thus, preferably, a composition according to the invention is not intended for the care and/or make-up of a keratinous material.

The invention also relates to a bottle provided with an application means and a packaging means, said packaging means containing a composition as described above.

A composition according to the invention is preferably topical.

This invention also relates to a non-therapeutic method of cosmetic treatment, in particular of perfuming a keratinous material or a garment, comprising at least one step of applying at least one composition according to the invention to the keratinous material and/or the said garment.

The present invention also relates to the use of at least one composition according to the invention to perfume a keratinous material or a garment.

The present invention also concerns the use of at least one composition according to the invention, to improve the persistence of the olfactory result of a perfume composition comprising less than 10%, preferably less than 5%, or even no alcohol(s), in particular ethanol.

The invention also relates to a cosmetic base, detergent or home fragrance comprising at least one composition as described above.

The present invention also relates to the use of at least one composition according to the invention to mask unpleasant odours.

The composition will preferably be applied/used by means of a spraying device (or means).

Throughout the description, the phrase “comprising a” is to be understood as synonymous with “comprising at least one”, unless otherwise specified. The terms “between . . . and . . . ”, “from . . . to . . . ” and “ranging from . . . to . . . ” shall be understood to include the low and high figures, unless otherwise specified.

### EXAMPLES

### Example 1: Comparative Study on the Continuous Phase

A comparative study is made on the 5 compositions described in the table below. These compositions are prepared using a non-microfluidic process with rotor/stator agitation.

TABLE 1

| COMPOSITION | | | | |
|---|---|---|---|---|
| A | B | C | D | E |
| Details | | | | |
| Inv. | Comp. | Comp. | Inv. | Inv. |

| | | | |
|---|---|---|---|
| Trade name | INCI name | % m in the | Phase |

Aqueous phase

| Osmosis water | Aqua | QSF* | | | | | A |
|---|---|---|---|---|---|---|---|
| Glycerine codex | Glycerine | 5 | | | | | A |
| Microcare | Pentylene Glycol | 2 | | | | | A |
| emollient PTG | | | | | | | |
| Microcare PE | Phenoxyethanol | 0.8 | | | | | A |
| Rheocrysta | Microcrystalline | 30 | 0 | 0 | 0 | 0 | B |
| C-2SN⁽¹⁾ | Cellulose (and) | | | | | | |
| | Phenoxyethanol | | | | | | |
| | (and) Water | | | | | | |
| Natrathix bio | Cellulose Gum | 0 | 1 | 0 | 0 | 0 | B |
| cellulose | | | | | | | |
| Kelcogel CG-HA | Gellan Gum | 0 | 0 | 0.2 | 0 | 0 | B |
| SENSOCEL | Microcrystalline | 0 | 0 | 0 | 1.5 | 0 | B |
| STAB 026 | Cellulose, Cellulose Gum | | | | | | |
| SENSOCEL | Xanthan Gum, | 0 | 0 | 0 | 0 | 1.5 | B |
| STAB 026-N | Microcrystalline Cellulose | | | | | | |

| | |
|---|---|
| Total | 100 |

Fatty phase (IF)

| | | | |
|---|---|---|---|
| Fragrance | — | QSF* | D |
| CAS-3131 | Amodimethicone | 0.2 | D |

| | |
|---|---|
| Total | 100 |

⁽¹⁾Rhéocrysta C-2SN contains 2% microcrystalline cellulose; the OF therefore contains 0.6% by weight of microcrystalline cellulose based on the weight of the OF

### Preparation Protocol:

Mix the raw materials A in a container with stirring at room temperature (=mix 1).
Add raw material B to mixture 1, stirring at room temperature until homogenised (=mixture 2).
At the same time, mix the raw materials D in a container with stirring at room temperature (=mix 3).
Add mixture 3 to mixture 2 (in a weight ratio of 12.2% IF/87.8% OF) with stirring at room temperature until homogenised.

The emulsions formed are then packaged in a 100 ml bottle fitted with a spray-type dispensing pump.

### Rating Criteria:

Using the table below, the aim is to obtain a composition with as high a total number of points as possible. A score of 0 for any parameter is eliminatory.

TABLE 2

| SCORING | | | |
|---|---|---|---|
| CRITERIA | 0 | 1 | 2 |
| Stability⁽²⁾ | Phase shift | Creaming | No phase shift |
| | OF and IF | IF rise but no | or creaming |
| | completely | coalescence | |
| | separate | | |
| Sprayability⁽³⁾ | Poor | Average | Good |
| | sprayability | sprayability | sprayability |
| | and/or delivery. | and/or | with uniform |
| | | inhomogeneous | delivery, |
| | | delivery. | |

⁽²⁾Stability is assessed by centrifugation; 80 ml of the composition to be tested is placed in a SIGMA centrifuge for 12 minutes at 3,500 rpm (radius approximately 13 cm).
⁽³⁾Good sprayability is characterised by delivery in the form of a cloud, and therefore the absence of a jet.

### Results

TABLE 3

| | A | B | C | D | E |
|---|---|---|---|---|---|
| Tests | Inv. | Comp. | Comp. | Inv. | Inv. |
| Stability | 1 | 0 | 1 | 2 | 2 |
| Sprayability | 2 | NA⁽⁵⁾ | 0 | 2 | 2 |
| High shear | 135 | NA⁽⁵⁾ | NA⁽⁵⁾ | 70 | 73 |
| viscosity⁽⁴⁾ | | | | | |
| Flow | 4.59 | NA⁽⁵⁾ | NA⁽⁵⁾ | 3.78 | 2.86 |
| threshold⁽⁴⁾ | | | | | |

⁽⁴⁾Viscosity and yield point are measured using the method described above.
⁽⁵⁾NA = Not Applicable as not assessed due to a stability and/or sprayability score of 0.

### Conclusion

The stability test for trial B led to release phenomena (separation of the two phases IF and OF) and was deemed unstable.

Test C is not satisfactory in terms of sprayability. Identical results to test C were obtained with a similar test (not described above) in which gellan gum was replaced by xanthan gum (Rhodicare T) at 0.5%.

Tests A, D and E relate to alcohol-free fragrance compositions that offer satisfactory sprayability while maintaining the stability and olfactory properties of the fragrance agent. These are the only compositions comprising at least microcrystalline cellulose.

These properties are further improved when the continuous aqueous phase also includes a natural gum (tests D and E), test D being even preferred to test E.

Trials A, D and E have excellent properties in terms of fragrance rendition and olfactory fidelity, and the persistence of the scented note over time after application. These A, D and E tests are also advantageous because they do not feel sticky to the touch.

Trials A′, D′ and E′ were conducted, the compositions of which differ from Trials A, D and E by the absence of amodimethicone. The results obtained in terms of stability and sprayability are slightly lower than those obtained with tests A, D and E, but are still satisfactory.

### Example 2: Comparative Study of the Fatty Phase

A comparative study is made on the 5 compositions described in the table below. These compositions are prepared by a non-microfluidic process using a Silversion.

TABLE 4

| COMPOSITION | | | | |
|---|---|---|---|---|
| F | G | H | I | J |
| Details | | | | |
| Comp. | Inv. | Inv. | Inv. | Inv. |

| | | | |
|---|---|---|---|
| Trade name | INCI name | % m in the | Phase |

Aqueous phase

| Osmosis water | Aqua | QSF* | | | | | A |
|---|---|---|---|---|---|---|---|
| Glycerine codex | Glycerine | 5 | | | | | A |
| Microcare | Pentylene Glycol | 2 | | | | | A |
| emollient PTG | | | | | | | |
| Microcare PE | Phenoxyethanol | 0.8 | | | | | A |
| VIVAPUR CS | Microcrystalline | 2 | | | | | B |
| TEX SUN | Cellulose (and) | | | | | | |
| | Cellulose Gum | | | | | | |
| CREMOPHOR GS32 | Polyglyceryl-3 | 0 | 0 | 0 | 3 | 0 | C |
| (ie non-ionic TA) | Distearate | | | | | | |

| | |
|---|---|
| Total | 100 |

Fatty phase (IF)

| Fragrance | — | QSF* | | | | | D |
|---|---|---|---|---|---|---|---|
| Eumulgin VL 75 | Lauryl Glucoside | 4 | 0 | 0 | 0 | 0 | D |
| (ie non-ionic TA) | (and) Polyglyceryl-2 | | | | | | |
| | Dipolyhydroxystearate | | | | | | |
| | (and) Glycerin | | | | | | |
| CAS-3131 | Amodimethicone | 0 | 0.2 | 0 | 0 | 0 | D |
| CosmeGreen ES1822+ | Arachidyl/Behenyl | 0 | 0 | 0.8 | 0 | 0 | D |
| (ie cationic TA) | Betainate Esylate | | | | | | |
| | (and) Arachidyl/ | | | | | | |
| | Behenyl Alcohol | | | | | | |
| CosmeGreen MB1618 | Cetearyl Alcohol | 0 | 0 | 0 | 0 | 0.5 | D |
| (ie TA cationic) | (and) Cetearyl | | | | | | |
| | Betainate Mesylate | | | | | | |
| Miglyol 829 eco | Caprylic/Capric/ | 0 | 0 | 0 | 0 | 2 | D |
| | Succinic Triglyceride | | | | | | |

| | |
|---|---|
| Total | 100 |

### Preparation Protocol:

Mix the raw materials A in a container with stirring at room temperature (=mix 1).
Add raw material B to mixture 1, stirring at room temperature until homogenised (=mixture 2).
When present, add raw material C to mixture 2, stirring in a bain-marie at 80° C. until homogenised (=mixture 3).
At the same time, if present, mix the raw material D in a container with stirring at room temperature (for tests F and G) or in a water bath at 80° C. (for tests H and J) (=mix 4).
Add mixture 4 to mixture 2 (mixture 3 for test I) (in a weight ratio of 12.2% IF/87.8% OF), stirring at room temperature until homogenised.

The emulsions formed are then packaged in a 100 ml bottle fitted with a spray-type dispensing pump.

Scoring criteria: see example 2.

### Results

TABLE 5

| | F | G | H | I | J |
|---|---|---|---|---|---|
| Tests | Comp. | Inv. | Inv. | Inv. | Inv. |
| Stability | 0 | 2 | 1 | 1 | 2 |
| Sprayability | NA⁽⁵⁾ | 2 | 2 | 2 | 2 |
| High shear | NA⁽⁵⁾ | 85 | 89 | 88 | 79 |
| viscosity⁽⁴⁾ | | | | | |
| Flow | NA⁽⁵⁾ | 4.06 | 4.73 | 3.57 | 4.04 |
| threshold⁽⁴⁾ | | | | | |

### Conclusion

The stability test for trial F led to release phenomena (separation of the two phases IF and OF) and was deemed unstable.

Tests G to J relate to alcohol-free fragrance compositions that offer satisfactory sprayability while maintaining the stability and olfactory properties of the fragrance agent.

Trials G to J have excellent properties in terms of fragrance rendition and olfactory fidelity, as well as the persistence of the scented note over time after application. These G to J tests are also advantageous because they do not feel sticky to the touch.

1. A composition, in the form of an oil-in-water emulsion comprising at least one fatty phase in the form of drops dispersed in a continuous aqueous phase, the fatty phase and the aqueous phase being immiscible with one another at room temperature and atmospheric pressure, characterised in that:
a) the continuous aqueous phase comprises at least microcrystalline cellulose, and
b) the dispersed fatty phase comprises at least 5% by weight of perfume(s) relative to the total weight of the composition.

2. The composition of claim 1, wherein the composition has a high shear viscosity, as measured at 25° C. and under a shear stress of 100 s⁻¹, of less than or equal to 150 mPa·s.

3. A composition according to claim 1, in which the composition is sprayable.

4. A composition according to claim 1, in which the continuous aqueous phase has a flow threshold value of less than or equal to 10 Pa.

5. A composition according to claim 1, in which the composition comprises from 0.1% to 10% by weight of microcrystalline cellulose relative to the total weight of the continuous aqueous phase.

6. A composition according to claim 1, in which the continuous aqueous phase additionally comprises at least one natural gum.

7. A composition according to claim 1, in which the at least one natural gum is chosen from cellulose, starch, xanthan, inulin, gellan, pullulan, curdlan, gum arabic, amylopectin, dextran, hemicellulose and mixtures thereof.

8. A composition according to claim 1, in which the composition comprises from 0.1% to 10% by weight of natural gum(s) relative to the total weight of the continuous aqueous phase.

9. A composition according to claim 1, in which the dispersed fatty phase comprises at least one lipophilic cationic compound comprising at least one primary, secondary, tertiary or quaternary amine function and/or the continuous aqueous phase comprises at least one non-ionic surfactant.

10. A composition according to claim 1, in which the lipophilic cationic compound comprising at least one primary, secondary, tertiary or quaternary amine function is chosen from at least one polymer modified with a primary, secondary, tertiary or quaternary amine function, a cationic surfactant, and mixtures thereof.

11. A composition according to claim 1, in which the composition comprises from 0.05% to 10% by weight of lipophilic cationic compound(s) comprising at least one primary, secondary, tertiary or quaternary amine function relative to the total weight of the dispersed fatty phase.

12. A composition according to claim 1, in which the composition comprises from 0.1% to 10% by weight of non-ionic surfactant(s), relative to the total weight of the continuous aqueous phase.

13. A composition according to claim 1, in which the fatty phase comprises from 10% to 100% by weight of perfume(s) relative to the total weight of the dispersed fatty phase.

14. A composition according claim 1, in which the fatty phase comprises from 5% to 30% by weight of perfume(s) relative to the total weight of the composition.

15. A composition according to claim 1, in which the composition comprises from 1% to 30% by weight of oil(s) relative to the total weight of the dispersed fatty phase.

16. A composition according to claim 1, in which the composition comprises from 5% to 50% by weight of fatty phase relative to the total weight of the composition.

17. A composition according to claim 1, in which the composition comprises less than 10% of alcohol(s), relative to the total weight of the composition.

18. Non-therapeutic process for cosmetic treatment, comprising a step of applying to the said keratinous material and/or the said garment at least one composition according to claim 1.

19. A method for perfuming a keratinous material or a garment comprising a step of applying to the said keratinous material and/or the said garment at least one composition according to claim 1.
